(19) United States
(12) Patent Application Publication
(10) Pub. No.: US 2007/0179608 A1
(43) Pub. Date: Aug. 2, 2007

(54) SYSTEM AND METHOD FOR ARTICULAR SURFACE REPAIR

(75) Inventors: Steven W. Ek, Bolton, MA (US); Anthony Miniaci, Bentleyville, OH (US); Carl Hasselman, Franklin, MA (US)

Correspondence Address: GROSSMAN, TUCKER, PERREAULT & PFLEGER, PLLC, 55 SOUTH COMMERICAL STREET, MANCHESTER, NH, 03101, US

(73) Assignee: ARTHROSURFACE, INC., Franklin, MA (US)

(21) Appl. No.: 11/461,240

(22) Filed: Jul. 31, 2006

Related U.S. Application Data
(60) Provisional application No. 60/703,852, filed on Jul. 29, 2005.

(51) Int. Cl. A61F 2/30

(52) U.S. Cl. 623/14.12; 623/23.39

(57) ABSTRACT

A system, method for replacing at least a portion of first articular surface cooperating with a second articular surface including a prosthetic implant. The method including creating a socket in the first articular surface generally opposite the prosthetic implant. A graft of biological material is installed in the socket, providing a biological replacement of a portion of the first articular surface.

### CROSS-REFERENCE TO RELATED APPLICATIONS

This application claims the benefit of U.S. provisional patent application Ser. No. 60/703,852, filed on Jul. 29, 2005, the entire disclosure of which is incorporated herein by reference.

### FIELD OF THE DISCLOSURE

The present disclosure relates to systems and methods for repairing and/or replacing at least a portion of an articular surface.

### BACKGROUND

Articular cartilage is tissue on the cooperating, or articulating, surfaces of bone in the body. Typically, articular cartilage is composed of hyaline cartilage, which has many unique properties that allow it to function effectively as a smooth and lubricious load-bearing surface. However, when injured, damaged hyaline cartilage cells are not typically replaced by new hyaline cartilage cells. Healing is dependent upon the occurrence of bleeding from the underlying bone and may often result in the formation of scar tissue or reparative cartilage called fibrocartilage. While similar to hyaline cartilage, fibrocartilage does not possess the same unique aspects of native hyaline cartilage and tends to be far less durable.

Hyaline cartilage problems, particularly in knee and hip joints, are generally caused by disease, such as rheumatoid arthritis, or by wear and tear (osteoarthritis). Damaged hyaline cartilage may also be the result of an injury, either acute, i.e., sudden injury, or recurrent and chronic, i.e., ongoing damaging activity. Such cartilage disease or deterioration can compromise the articular surface causing pain and further deterioration of hyaline cartilage and a loss, diminution, of joint function. As a result, various methods have been developed to treat and repair damaged or destroyed articular cartilage.

### BRIEF DESCRIPTION OF THE DRAWINGS

Features and advantages of the invention herein will be apparent from the following description of embodiments consistent therewith, which description should be considered in conjunction with the accompanying drawings, wherein:

FIG. 1 schematically depicts a joint including an implant associated with one articular surface and a defect associated with a second, cooperating articular surface;

FIG. 2 illustrates the joint shown in FIG. 1 with a portion of the second articular surface excised to provide a socket;

FIG. 3 shows the joint of FIG. 2 with a tissue graft installed in the socket of the second articular surface;

FIG. 4 depicts a joint including a second articular surface having a socket formed therein and a tissue graft suitable for installation into the socket;

FIG. 5 illustrates an exploded cross-sectional view of a first embodiment of a graft consistent with the present disclosure;

FIG. 6 is a cross-sectional view of the first embodiment of a graft in an assembled condition;

FIG. 7 shows another embodiment of a graft consistent with the present disclosure;

FIG. 8 is a cross-sectional view of yet another embodiment of a graft the consistent with the present disclosure; and

FIG. 9 depicts, in cross-sectional view, still another embodiment of a consistent with the present disclosure.

### DESCRIPTION

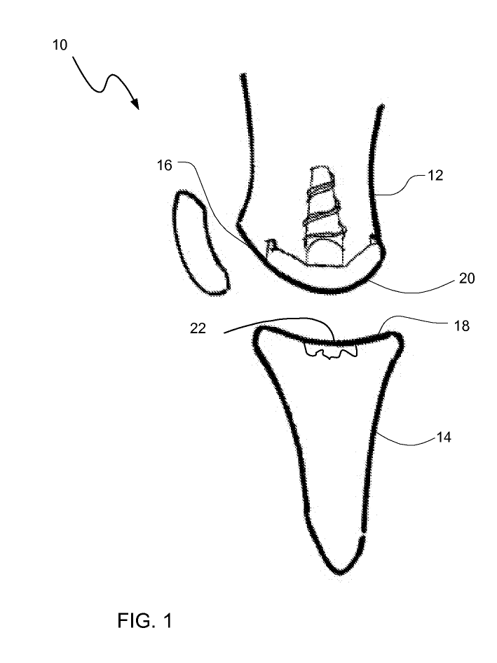

Referring to FIG. 1, an exemplary joint 10 is schematically depicted. As shown, the joint 10 may include a first and second bone 12, 14 including respective first and second articular surfaces 16, 18. The first and second articular surfaces 16, 18 may cooperate to provide smooth articulation of the bones 12, 14 relative to one another. As shown, the first articular surface 16 may include a repair component in the form of a prosthetic implant 20, which may replace at least a portion of the articular surface 16. Examples of suitable implants for replacing at least a portion of an articular surface are described, for example, in U.S. Pat. No. 6,520,964 and U.S. Pat. No. 6,679,917, the entire disclosures of which are incorporated herein by reference. Consistent with such exemplary implants, a prosthetic implant may include a biologically compatible material, such as a biocompatible metal, polymer, or ceramic. The prosthetic implant may be installed in a socket formed in the first articular surface, such that the prosthetic implant may be at least partially disposed in the first articular surface. The prosthetic implant may have a load bearing surface that is configured to replace at least a portion of the first articular surface of the first bone. In particular embodiments, the load bearing surface of the implant may have a geometry that is based on the geometry of the portion of the articular surface being replaced. Of course, implants having various other configurations may also suitably be employed for replacing at least a portion of one articular surface in connection with the present disclosure.

The implant 20 may be provided replacing a defective portion of the first articular surface 16, such as a damaged or diseased portion of the articular surface 16. In general, the implant 20 replacing at least a portion of the first articular surface 16 may be configured to provide suitable articulation relative to, and in conjunction with, the second articular surface 18. However, in some instances the second articular surface 18 may include a damaged region 22 which may impair, or reduce, the operation and articulation of the joint 10. The damaged region 22 of the second articular surface 18 may be the result of injury, such as the injury which necessitated the replacement of a portion of the first articular surface 16 with the implant 20. In other instances, the damaged region 22 of the second articular surface 18 may be due to disease, be the result of interaction between the implant 20 and the second articular surface 18, etc.

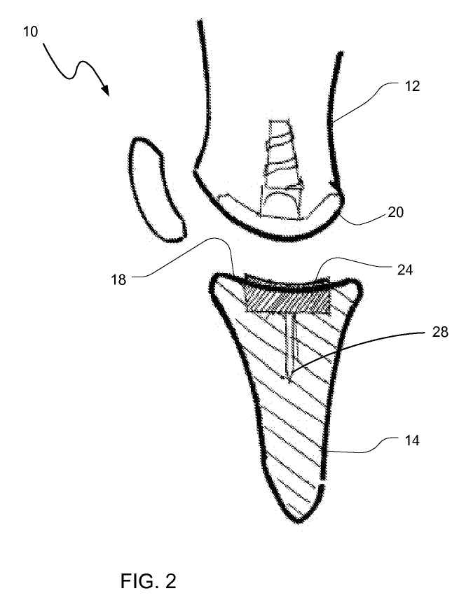

Turning to FIG. 2, the damaged region 22 of the second articular surface 18 opposite to, and possibly at least partially cooperating with, the implant 20 replacing at least a portion of the first articular surface 16 may be repaired using a graft 24 of biological material. The graft 24 of biological material may replace at least a portion of the second articular surface 18, or may otherwise at least partially repair at least a portion of the damaged region 22. During articulation of the joint, the graft 24 of biological material may interact with the first articular surface 16, which may, in part, include the implant 20. The interaction between the graft 24 of biological material and the implant 20 or first articular surface 16 may provide suitably smooth articulation and/or function of the joint 10.

Consistent with the present disclosure the graft 24 may include a variety of biological materials. Suitable biological materials may include living human tissue autografts composed of fibrous or connective tissues. Examples of suitable fibrous or connective tissues which may be used for grafts of biological material may include joint capsular tissues, tendons or tendon sheaths, human allograft tissues, etc. Such tissues may be harvested, for example, from regions surrounding an operative site created to repair and/or replace at least a portion of the first and/or second articular surfaces, and/or may otherwise be provided.

In addition to directly harvested tissue, materials derived from human tissues may also be employed for producing suitable grafts of biological materials. For example, one suitable material derived or manufactured from human tissue may be an acellular scaffold which may include one or more of collagen and extra-cellular protein matrices, e.g., composed of elastin, collagen, proteoglycan, and preserved blood vessel channels derived from human dermal tissues. An example of a commercially available manufactured material is available as GraftJacket, which is available from Wright Medical Technology. Other similar tissues derived from bovine or equine sources, such as hides, pericardium, and other tissue types, may also be employed herein. Various other natural, manufactured, or derived tissues and biological material may also suitably be employed as grafts consistent with the present disclosure.

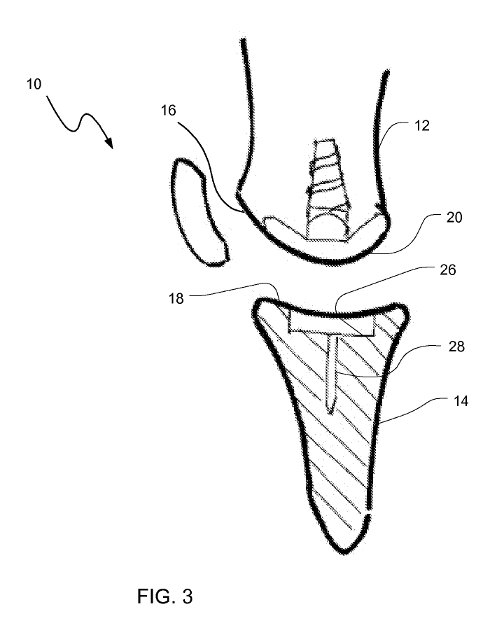

As shown in FIG. 3, the second articular surface 18 may be prepared for receiving a graft 24 of biological material by providing a socket 26 in the second articular surface 18. The socket 26 may be positioned at least partially in the region of the damaged region 22 of the second articular surface 18. In one embodiment, the socket 26 may be sized to remove at least a portion, if not all, of the damaged region 22 of the second articular surface. In such an embodiment, at least a portion of the damaged region 22 of the second articular surface 18 may be replaced by the graft 24 of biological material.

The socket 26 in the second articular surface 18 may be prepared using a variety of techniques. According to one such technique, a guide pin may be installed extending at least partially into the second articular surface 18. A socket may be created around the guide pin using a cutter that is rotatable about the guide pin for excising a circular region of the articular surface centered on the guide pin. The guide pin may be positioned in a hole 28 extending at least partially into the second articular surface 18. The hole 28 may be drilled into the second articular surface 18 and the guide pin may be inserted into the hole 28. Alternatively, the guide pin may, itself, be drilled into the second articular surface 18. Consistent with the latter technique, preparing the hole 28 and inserting the guide pin therein may be achieved using a single step process. A method of forming a socket generally consistent with the foregoing description is described in U.S. Pat. No. 6,520,964, the disclosure of which is incorporated herein by reference.

Various techniques may be used to achieve desired positioning and orientation of the guide pin extending at least partially into the articular surface. In a basic procedure, the placement and orientation of the guide pin may be achieved through visual inspection. The hole 28 may be created and the guide pin may be positioned extending at least partially into the second articular surface 18 using free-hand manipulation of the relevant instruments and components. According to an alternative embodiment, one or more guides, such as a drill guide, may be used to achieve a desired placement and orientation of the guide pin relative to the second articular surface.

As mentioned above, once the guide pin has been positioned extending at least partially into the second articular surface 18, a rotary cutter or cannulated drill may be positioned over the guide pin. The rotary cutter may be rotationally driven into the second articular surface to excise the socket 26. The rotary cutter or cannulated drill may be guided into the second articular surface 18 by the guide pin. In such a manner, the location and orientation of the socket 26 may be based on the location and orientation of the guide pin relative to the second articular surface 18.

Various additional and/or alternative techniques may also be employed for preparing a socket suitable for receiving a graft of biological material consistent with the present disclosure. For example, a cutter, drill, reamer, or the like may be used to excise a socket in the second articular surface with and/or without using a guide pin to direct and/or control the excision. Similarly, a rasp or other scraping instrument may be used to scrape or abrade a portion of the second articular surface corresponding to the desired site for the graft of biological material.

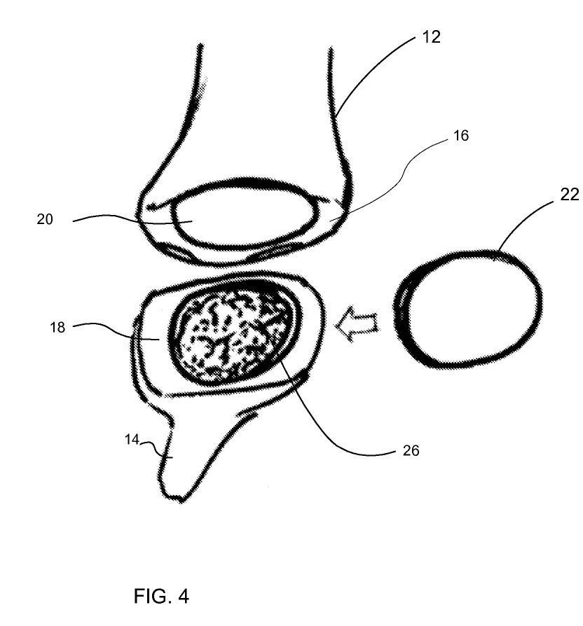

Turning next to FIG. 4, with the socket 26 prepared in the second articular surface 18, the graft of biological material 22 may be delivered to the socket 26. The graft of biological material 22 may be at least partially inserted into the socket 26 and may be secured to the second bone 14, and or the second articular surface 18. A variety of techniques and configurations of the graft of biological material 22 may be employed to secure the graft 24 in position relative to the second articular surface 18.

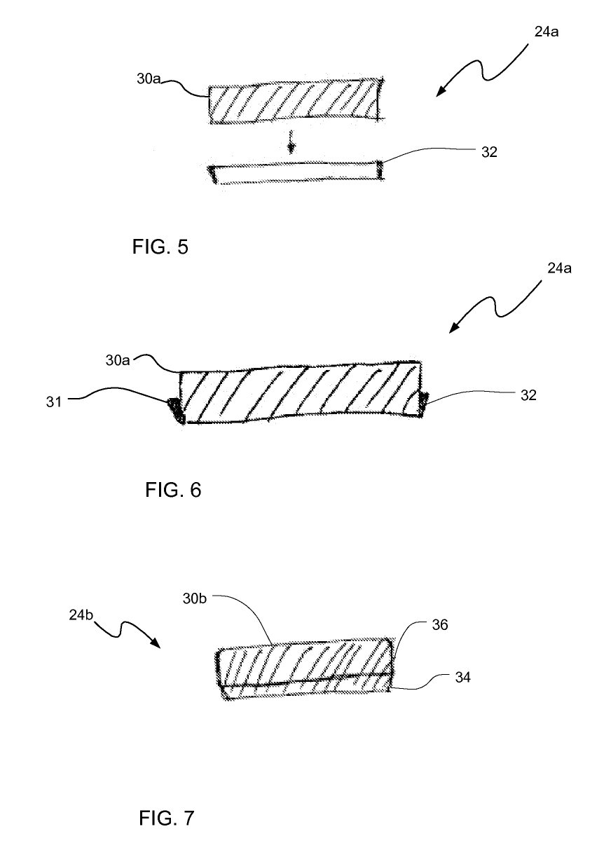

FIG. 5 depicts one embodiment of a graft 24a which may suitably be used for repairing a damaged portion of an articular surface opposed to a prosthetic implant. As shown, the graft 24a may include a plug 30a of biological material and a band 32 or ring sized to receive at least a portion of the plug 30a of biological material. The band 32 may be formed from a biologically compatible metallic, polymeric, ceramic, etc., material. As shown in FIG. 6, the plug 30a of biological material may be pressed into the band 32 to form the graft 24a. According to one embodiment, the band 32 may have a height less than the thickness of the plug 30a. Accordingly, in an installed condition the band 32 may not contact a cooperating articular surface 16 and/or prosthetic implant 20. In one embodiment, the band 32 may be crimped onto the plug 30a, thereby securing the band 32 to the plug 30a to resist separation of the plug 30a and the band 32.

The graft 24a may be pressed into the socket 26 and the band 32 and/or the plug 30a may engage a peripheral wall of the socket 26. Engagement between the band 32 and the peripheral wall of the socket 26 may resist separation of the graft 24a and the second bone 14. As shown in FIG. 6, according to one embodiment the band 32 may present an outward projection 31, as by having a tapered cross-section. The outward projection 31 of the band 32 may facilitate engagement with the peripheral wall of the socket 26 and may promote retention of the graft 24a in the socket 26. Additionally and/or alternatively, the band 32 may increase the radial compressive rigidity of the graft 24a. An increase in the radial compressive rigidity of the graft 24a may additionally facilitate retention of the graft 24a in the socket 26, and/or may decrease deformation and/or distortion of the plug 30a.

Another embodiment of a graft 24b consistent with the present disclosure is shown in FIG. 7. The graft 24b may include a unitary plug 30b of biological material. Consistent with such an embodiment, the plug 30b of biological material may be sized to provide a press or interference fit with the socket 26. For example, the plug 30b may be oversized relative to the socket 26, such that the frictional engagement between the plug 30b and the socket 26 may resist separation of the graft 24b and the second bone 14.

Installation of the plug 30b into the socket 26 may be facilitated by providing a taper 34 or chamfer on a lower portion of the sidewall 36 of the plug 30b. According to one embodiment, the taper 34 may provide a portion of the plug 30b having a diameter less than a diameter of the socket 26. In such an embodiment, the taper 34 may provide a lead in which may facilitate insertion of the plug 30b into the socket 26. Additionally and/or alternatively, the taper 34 may facilitate alignment between the graft 24b and the socket 26, and/or accommodate any initial misalignment between the graft 24b and the socket 26.

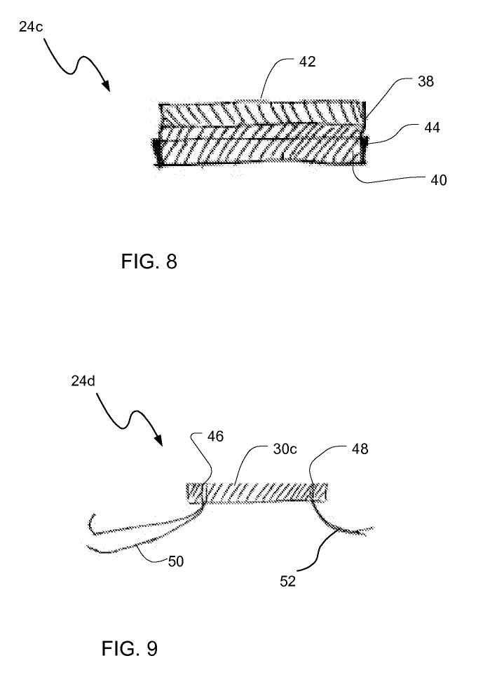

Turning to FIG. 8, yet another embodiment of a graft 24c is illustrated. The graft 24c may be provided as a multi-layered structure. For example, the graft 24c may include a top layer 38 and a bottom layer 40. In one embodiment, the bottom layer 40 may be more rigid than the top layer 38. The higher rigidity of the bottom layer 40 may facilitate handling of the graft 24c and/or may prevent or reduce deformation and/or distortion of the graft 24c. The top layer 38 may exhibit a low coefficient of friction. As such, the graft 24c may have sufficient rigidity to allow facile handling and may provide a lubricious top surface 42 for interacting with a cooperating implant 20. According to various embodiments, the top layer 38 and the bottom layer 40 may each include a biological material. Alternatively, the top layer 38 and/or the bottom layer 40 may include a non-biological material, such as a polymeric material, a metallic material, a ceramic material, etc. In further embodiments, one and/or both of the top layer 38 and the bottom layer 40 may include a combination of biological materials and non-biological materials.

According to various embodiments, the lubricious top layer 38 and the comparatively rigid bottom layer 40 may be formed as separate layers. The separate top 38 and bottom layers 40 may be coupled to one another, either directly or indirectly. For example, the top 38 and bottom 40 layers may be adhesively bonded. In a related embodiment, the graft 24c may be a molded product in which the top 38 and bottom 40 layers may be molded on to one another. For example, the bottom layer 40 may be molded first and then the top layer 38 may be directly molded on the bottom layer 40, thereby joining the top 38 and bottom 40 layers. Other methods of joining the top and bottom layers may also suitably be employed herein. Additionally, the graft 24c may be provided as a multi-layered structure including additional layers. According to another embodiment, the graft may be provided as a unitary body having characteristics of rigidity and/or lubricity that may vary across the thickness of the graft. For example, the lubricity of the graft may increase toward the top surface and the rigidity of the graft may increase away from the top surface. Consistent with such an embodiment, the graft need not include discrete layers. The graft may be a unitary member including comparatively rigid regions and comparatively lubricious regions.

As shown, the graft 24c may include features to aid in retaining the graft 24c in the socket 26. For example, the graft 24c may include a ridge 44 disposed around at least a portion of the perimeter of the graft 24c. The ridge 44 may be tapered inwardly toward the bottom of the graft 24c. Consistent with the previously described structure of the graft 24c, the ridge 44 may extend from the bottom layer 40 of the graft 24c. As such, the ridge 44 may be at least partially received in the socket 26 when the graft 24c is installed therein. In one embodiment, the ridge 44 may be integrally formed with the graft 24c, such as an integrally molded feature of the graft 24c. As such, in an embodiment including a comparatively rigid bottom layer 40, the ridge 44 may similarly be comparatively rigid. Engagement between the ridge 44 and the socket 26 may resist separation of the graft

FIG. 9 shows still another embodiment of a graft 24d according to the present disclosure. Similar to the embodiments previously discussed with reference to FIGS. 5 through 7, the graft 24d may generally include a plug 30c of biological material. The plug 30c may include one or more suture features. As shown, the suture features may include openings 46, 48 in the plug 30c. The openings 46, 48 may provide convenient features for attaching sutures 50, 52. The sutures 50, 52 may allow the graft 24d to be secured in the socket 26 via sutures. Various additional or alternative suture features may also suitably be employed consistent with this aspect of the disclosure.

The various embodiments of a graft of biological material suitable for use consistent with the system for repairing and/or replacing at least a portion of an articular surface may include features or aspects for securing the graft in position relative to a socket formed in the second articular surface and/or for resisting separation of the graft from the second articular surface, e.g., for resisting separation and/or removal of the graft from the socket. In addition to including features or aspects for securing and/or retaining the graft relative the second articular surface, an embodiment of a graft herein may be used in conjunction with other methods and means for affixing, retaining and/or securing the graft relative to the second articular surface, e.g. cement, etc. According to any embodiment herein, the graft of biological material may be at least partially integrate and/or attach to native tissue in which the socket is formed and/or surrounding the socket. Accordingly, installation techniques and retention techniques and features may retain the implant in position within the socket for a sufficient period to permit biological interaction/integration between the graft and the native tissue surround and/or defining the socket.

Consistent with the foregoing, the present disclosure may generally provide a system, method and various apparatuses for replacing at least a portion of an articular surface, such as an articular surface opposing an articular surface implant, with a graft of biological material. According to one embodiment, a method consistent with the present disclosure may include forming a socket in a first articular surface opposite an implant replacing at least a portion of a second articular surface. The method may further include installing a graft of biological material into the socket.

According to a first aspect, the present disclosure may provide a method for replacing a portion of an articular surface. The method may include forming a prosthetic implant, in which the prosthetic implant is configured to be at least partially received in a first articular surface. The method may further include forming a biological graft, which is configured to be at least partially received in a second articular surface. The second articular surface may be in cooperating association with the first articular surface which is configured to at least partially receive the prosthetic implant.

According to another aspect, the present disclosure may provide a system for repairing a first and second cooperating articular surface. The system may generally include a prosthetic implant that is configured to replace at least a portion of the first articular surface. The system may further include a graft of biological material that is configured to replace at least a portion of said second articular surface cooperating with, at least in part, the prosthetic implant.

The various embodiments described herein have been presented for the purpose of illustrating various aspects, features, and advantages of the present invention. As such, the described embodiments should not be construed as limiting the scope of the present invention. Additionally, while numerous embodiments including various aspects and features have been separately described, it should be appreciated that the features and aspects of the several embodiments are susceptible to combination with one another.

What is claimed is:

1. A method for replacing a portion of an articular surface comprising:
forming a prosthetic implant, said prosthetic implant configured to be at least partially received in a first articular surface; and
forming a biological graft configured to be at least partially received in a second articular surface, said second articular surface cooperating with said first articular surface configured to receive said prosthetic implant.

2. The method according to claim 1, wherein said graft of biological material comprises a living human tissue autograft.

3. The method according to claim 1, wherein said graft of biological material comprises a living human tissue autograft comprising joint capsular tissue.

4. The method according to claim 1, wherein said graft of biological material comprises a living human tissue autograft comprising tendons or tendon sheaths.

5. The method according to claim 1, wherein said graft of biological material comprises a living human tissue allograft.

6. The method according to claim 1, wherein said graft of biological material comprises materials derived from human tissue.

7. The method according to claim 1, wherein said graft of biological material comprises an acellular scaffold derived from human tissue.

8. The method according to claim 1, wherein said biological material comprises tissue derived from bovine or equine sources.

9. The method according to claim 1, further comprising forming a socket in said second articular surface for receiving said biological implant, comprising resecting a portion of said second articular surface comprising a damaged region opposite said prosthetic implant.

10. The method according to claim 9, further comprising anchoring said graft of biological material in said socket.

11. A system for repairing a first and second cooperating articular surface, the system comprising:
a prosthetic implant configured to replace at least a portion of said first articular surface; and
a graft of biological material configured to replace at least a portion of said second articular surface cooperating with, at least in part, said prosthetic implant.

12. A system according to claim 11, wherein said graft of biological material comprises a living human autograft.

13. A system according to claim 11, wherein said graft of biological material comprises a living human tissue allograft.

14. A system according to claim 11, wherein said graft of biological material comprises tissue derived from human tissue.

15. A system according to claim 11, wherein said graft of biological material comprises tissue derived from bovine or equine sources.

16. A system according to claim 11, wherein said graft of biological material comprises a plug of biological material and a ring configured to receive at least a portion of said plug.

17. A system according to claim 16, wherein said ring is crimped onto said plug of biological material.

18. A system according to claim 16, wherein said ring comprises an outward projection configured to promote retention of said graft in a socket in said second articular surface.

19. A system according to claim 11, wherein said graft of biological material comprises a multi-layer structure, wherein at least a top layer comprises a biological material.

20. A system according to claim 11, wherein said graft of biological material comprises at least one ridge extending around at least a portion of a perimeter of said graft, said ridges configured to promote retention of said graft in a socket in said second articular surface.
